(19) United States
(12) Patent Application Publication
(10) Pub. No.: US 2016/0213333 A1
(43) Pub. Date: Jul. 28, 2016

(54) DIAGNOSTIC DIGITAL DATA MINING OF BIOLOGICAL WAVES WITH SPECTRAL ELECTROCARDIOGRAPHY (SECG)

(71) Applicants: Juan R. GUERRERO, Kuenzing, Lower Bavaria (DE); Robin LOBEL, Ivry sur Seine (FR)

(72) Inventors: Juan R. GUERRERO, Kuenzing, Lower Bavaria (DE); Robin LOBEL, Ivry sur Seine (FR)

(21) Appl. No.: 14/917,655

(22) PCT Filed: Sep. 10, 2014
(86) PCT No.: PCT/US2014/054890

§ 371 Date: Mar. 9, 2016

Related U.S. Application Data
(60) Provisional application No. 61/875,890, filed on Sep. 10, 2013.
(60) Provisional application No. 62/048,059, filed on Sep. 9, 2014.

(51) Int. Cl. A61B 5/00; A61B 5/0476; A61B 5/021; A61B 5/024; A61B 5/145; A61B 5/0402; A61B 5/0488

(52) U.S. Cl. CPC A61B 5/742; A61B 5/0402; A61B 5/0476; A61B 5/0488; A61B 5/02416; A61B 5/14542; A61B 5/021

(57) ABSTRACT

Systems, methods and computer-readable media are provided to enable early diagnosis of a wide variety of potentially lethal or catastrophic medical conditions using improved diagnostic analysis of non-stationary, non-linear biological signals. The analysis techniques includes accessing a biological signal in the memory; determining a region of a recording to be displayed for the accessed biological signal; obtaining a signal of a morphologic characteristic and a signal of a spectral frequency characteristic included in the accessed biological signal; displaying for the determined region of the recording, spatially-separated and synchronized in time with each other, a first visually-compressed view including the signal of the morphologic characteristic and a second visually-compressed view including the signal of the spectral frequency characteristic; and, upon receiving an input from a user, changing a degree of visual compression of the displayed first visually-compressed view and displayed second visually-compressed view.

### CROSS-REFERENCE TO RELATED APPLICATIONS

This application is a national phase application under 35 U.S.C. §371 of international application no. PCT/US2014/054890 filed on Sep. 10, 2014, which designates the U.S., and which claims the benefit of U.S. Provisional Patent Application No. 61/875,890 filed on Sep. 10, 2013, and U.S. Provisional Patent Application No. 62/048,059 filed on Sep. 9, 2014. Each of the applications PCT/US2014/054890, 61/875,890, and 62/048,059 is hereby incorporated by reference in its entirety.

### TECHNICAL FIELD

The disclosure relates generally to systems and methods for the processing and analysis of short or long term biological signals such as, for example, electrocardiogram (ECG), electro-retinography (ERG), electroencephalogram (EEG), electromyogram (EMG), ambulatory blood pressure (ABPM), photoplethysmography, oxymetry, and phonocardiography.

### BACKGROUND AND SUMMARY

Non-linear dynamics are preponderant in biology. Biological signals are typically irregular, non-linear and non-stationary (Ary L. Goldberger, “Complex Systems,” Proc. Am. Thor. Soc. vol. 3 pp 467-472, 2006). Physiologic systems in health and disease display an extraordinary range of temporal behaviors and structural patterns that defy understanding when being viewed and/or analyzed using linear constructs, reductionist strategies, and classical homeostasis principles. Transient behavior of cardiovascular variables may emerge from the recovery of the system after a severe disturbance or from adaptive behavior throughout changes of physiologic and pathologic states. (A. Mueller et al., “Coupling analysis of transient cardiovascular dynamics,” Biomed. Tech. 2013, 58, pp 131-139).

Even the simplest non-linear dynamic systems contravene the proportionality and superposition that characterize linear systems. Proportionality results in output that bears a straight-line relationship to the input. Superposition refers to the aspect that the output of linear systems, composed by multiple components, can be fully understood and predicted. Non-linear systems lack proportionality, and cannot be superimposed or predicted. Proportionality does not hold for non-linear systems; small changes in the signal can have dramatic consequences that cannot be predicted.

Non-linear systems are composed of multiple subunits that need to be identified and separated to be analyzed as individual modular components of a global system. Non-linear coupling have consequences that cannot be explained and predicted if traditional analytical methods are applied. However, although non-linear systems may differ in specific details, they have certain common patterns of response. For example, non-linear systems are characterized by abrupt appearances of unanticipated effects that result from complex interactions; a minor change in a parameter may cause an abrupt and disproportionate change in degree and nature of the consequence.

Non-stationarity is common in biomedical time series, such as heart beats, where normal beats may be interrupted by singular, potentially malignant, events. The unpredictable origin of unforeseen events render non-stationary signals such as ECG unsuitable for standard statistical analytical methodology used in conventional systems.

The non-stationarity and non-linearity characteristics of biological signal series are an obstacle for digital analysis with the conventional tools in use today, which is a reason for the inaccuracy and diagnostic failure of current digital analysis of biomedical series with the plethora of false negative results of current ECG signal analysis methodology. (Millis Hurst, M. D., “Current status of Clinical Electrocardiography with suggestions for the improvement of the interpretative process,” The American Journal of Cardiology, Vol 92, pp. 1072-1079, November 2003; C. M. Yong et al., “The Electrocardiogram at a Crossroads,” Circulation 2013, 128:7982; N. A. Mark Estes III., “Computerized Interpretation of ECGs, Supplement Not a Substitute,” Circ. Arrhythm. Electrophysiol., 6:2-4, 2013).

Therefore, alternative user-friendly cost-effective methodologies are needed to identify (e.g., by data mining) and extract biological signals that portend risk for potentially lethal or catastrophic conditions for efficacious preventive therapeutic intervention.

The ECG is the conventional tool for testing the integrity of the life-sustaining electrical function of the heart. The ECG wave of today is the instantaneous visualization of all simultaneously occurring electrical currents in the heart plus contaminating noise. Spectral ECG (SECG), described in relation to the embodiments of the invention disclosed herein, makes it possible for the first time since 1902 to separate, and to make visible for analysis, the individual noise-free frequency bands free of the contamination and distortion caused by frequencies of no interest. The signal of all simultaneously occurring electrical currents in the heart rate are hidden and invisible within the traditional ECG thin line that represents the instantaneous mean vector of all the simultaneous electrical activity in the myocardium. The inventors believe that SECG, according to embodiments, transforms conventional ECG analysis to a level similar to how Computerized Axial Tomography (CAT scan) transformed the X-Ray analysis. SECG is a disruptive innovation that can save lives and diminish disability by enabling non-invasive electrophysiology to be used in preventive cardiology. SECG provides for early detection of risk for serious cardiovascular conditions not possible with conventional digital ECG. A summary of some of the features of embodiments of the invention is provided below in this section.

An example embodiment includes computer-implemented data mining to improve medical diagnosis of a non-stationary non-linear biological signal acquired via one or more probes attached to a patient and stored in a memory. The method includes accessing an optimally recorded and processed biological signal to identify the signals within the all-inclusive signal. Novel spectral analysis now made possible with SECG enable identification of baseline and new onset frequencies which in conventional systems are embedded and rendered invisible in the conventional ECG. Heretofore, only morphology (e.g., interpretation of shape such as amplitude, width and contour) and metrology (e.g., measurement of voltage and duration of wave and interval in the ECG) of the all-inclusive single line that depicts the instantaneous mean vector of electrical activation of the heart is possible.

For each ECG lead recorded, SECG simultaneously displays all, or a plurality of the frequencies of all, the signals embedded within the traditional signal. Such singular, sub signals originate in the growing number of ionic pumps within the myocardial cell that govern the electrogenic and electrotonic life sustaining function of the heart.

The novel, fundamental, tool for ECG data mining with SECG, as disclosed herein, includes the deconstruction, separation and evaluation of the multiple frequency bands contained, and invisible, within the traditional ECG signal. The isolated frequency bands can be evaluated as single beats or as aggregated thousands of heart beats, as well as in a bi-dimensional or tri-dimensional mode. The onset, offset, morphology, power change within the frequency band etc., can be analyzed. In some example embodiments, the analysis includes visual analysis.

Upon opening of the recording in a visually compressed mode, the screen shows up to a very large number (e.g., hundreds of thousands) of heart beats in both the compressed morphology and frequency pattern panels. For example, a first view may include a visually compressed morphology and a simultaneously displayed second view may include visually compressed frequency patterns corresponding to the morphology. The visual display of the frequency pattern can be characteristic for certain pathologic conditions which can immediately lead the operator to narrow the diagnostic possibilities. These characteristics and patterns can be stored in a proprietary library of pathologic and normal patterns. The proprietary library of SECG patterns reside in the analytical devices, can be shared through the Internet to aid in the understanding and diagnostic use of SECG, in educational activities etc.

Stability or progressive changes in the range (e.g., in Hertz) of frequencies of interest help lead the operator to the most benign (i.e., basal, most normal ECG for the patient in a recording) as well as the areas of the recording where the greatest pathology is to be found.

The operator then proceeds to visually decompress the baseline and most pathologic areas of the recording for further visual morphologic and metrologic evaluation which permit reaching a range of diagnostic possibilities within a few minutes after (e.g. less than 10 minutes) opening long (e.g., 24 hrs. or longer) ECG recordings.

SECG enables single or simultaneous multiple (e.g., electrocardiogram, heart sounds, oximetry, electro mechanical coupling, etc.) wave spectral and morphologic analysis of non-stationary non-linear biological signals and permits instantaneous in depth analysis of supra-additive combinations of all detected single or multiple, normal or pathologic, waves for diagnostic purposes.

For medical diagnosis it is best to evaluate biological signals for a long period of time to include all possible forms of physical, emotional, physiological stress. SECG recording should preferably start with a 15 minutes period at the physician office in which standardized form of stress are to be induced. The ECG response to the challenge is to determine if the ambulatory recording is to be continued for at least 24 hours. The information thus obtained is far more valuable than that rendered by the commonly used 12 lead ECG at rest in which only 3 or 4 (frequently chosen at random by the computer) of the more than 120,000 heart beats we have in 24 hours are used for diagnosis. The 12 lead ECG at rest is a woefully under representative sample of reality that leads to false negative reports (the primary serious adverse event of the conventional ECG as currently done) by failing to document unpredictable, short pathologic episodes which is the way serious diseases usually start.

The recordings obtained from each electrode or signal sensing device (e.g. microphone, oxymetry etc.) are displayed in two synchronized panels showing the morphology (time/voltage wave) and the spectral frequency display. The frequency display can be of single beats or visually compressed thousands of beats. Displays can be bi- or tri-dimensional. Magnification of each panel can be controlled at will to facilitate diagnostic evaluation.

Unlike what has been used before, signal averaging or deleation of abnormal beats is not necessary. Signal averaging after exclusion of abnormal beats is unnecessary and misleading when dealing with non-stationary, non-linear signals; hence, it is avoided in embodiments of the present invention.

The biological signal may include, but is not limited to, at least one of electrocardiogram (ECG) signal, electroencephalogram (EEG) signal, electromyogram (EMG) signal, electro-retinography (ERG) signal, signal corresponding to respiratory functions, signal corresponding to ambulatory blood pressure (ABPM), signal generated by photoplethysmography, signal generated by oxymetry, or signal generated by phonocardiography.

Power variations in each frequency may be displayed using color bands that change in luminance, tonality, and saturation as the power level within the selected frequency band changes.

Selected frequency bands of interest may be displayed in configurable color and/or contrast in order to identify their respective contributions to the classical morphologic structure of the biological signal.

The medical diagnosis method may further include annotating one or more of the displayed first view and the displayed second view (e.g., at least partially decompressed views) with markers and text to identify waves or wave components of diagnostic interest.

The medical diagnosis method permits instantaneous shifting between different levels of visual compression and/or biological signal recording leads for said displaying of the first view and the second view.

The medical diagnosis method may provide static or dynamic screenshots of the display screen captured when displaying the first view and the second view of selected regions of interest in the recordings.

At least one of the first view or the second view may be displayed in three-dimensions facilitating fast and accurate finding of certain pathologic elements in the signal examined.

The medical diagnosis method provides for displaying automatically or manually identified normal or de novo abnormal transient and permanent frequencies the morphology of which are hidden and invisible within the traditionally acquired biological signal.

The medical diagnosis method may include displaying segregated frequencies of diagnostic interest. The segregated frequencies may be color coded for identification of their respective contributions to the morphology of the biological signal.

The medical diagnosis method may include comparison of at least one of the displayed first visually-compressed or visually-decompressed view with the displayed second view having a similar degree of compression and/or with a library of predetermined and evolving patterns to guide and speed up the differential diagnosis of possible pathology.

Another embodiment includes a medical diagnosis system to analyze a non-stationary non-linear biological signal acquired via one or more probes attached to a patient. The system includes a memory configured to store the acquired biological signal; and at least one processor. The processor may be configured to perform operations comprising: accessing the stored biological signal in the memory; determining a region of the recording to be displayed for the accessed biological signal; obtaining a signal of a morphologic characteristic and a signal of a spectral frequency characteristic included in the accessed biological signal; displaying for the determined region of the recording, spatially-separated and synchronized in time with each other, a first visually-compressed view including the signal of the morphologic characteristic and a second visually-compressed view including the signal of the spectral frequency characteristic; and upon receiving an input from a user, quickly changing a degree of visual compression of the displayed regions of the recording chosen to facilitate morphological evaluation.

The system may further include a display screen, wherein the first view and the second view are displayed via the display screen. The first view and/or the second view may be displayed with some level of decompression.

The system may also include a recorder electrically connected to the one or more probes and configured to acquire the biological signal.

Yet another embodiment includes a non-transitory computer-readable storage medium storing a computer program for analyzing, for medical diagnosis, a non-stationary non-linear biological signal acquired via one or more probes attached to a patient. The computer program, when executed by a processor, causes the processor to perform operations including: accessing the stored biological signal in the memory; determining region of the recording to be displayed for the accessed biological signal; obtaining a signal of a morphologic characteristic and a signal of a spectral frequency characteristic included in the accessed biological signal; displaying the regions spatially-separated but synchronized in time with each other, a first visually-compressed view including the signal of the morphologic characteristic and a second visually-compressed view for further evaluation.

The selection of frequencies of interest isolated in the certain regions of the recordings can be done manually (i.e., following the visually identified frequency band) while the left mouse button is compressed or automatically for which the initial identification can be done by hand and from then on the automated computerized instructions will further identify the selected frequency this feature is being developed to be activated while the signal streams in real time this feature is planned to be used in the design of automated failsafe alarms.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

These and other features and advantages may be better and more completely understood by reference to the following detailed description of exemplary illustrative embodiments in conjunction with the drawings, of which:

FIG. 1 illustrates a block diagram of a system for diagnostic data mining of diverse biological waves, in accordance with certain example embodiments;

FIG. 2 illustrates a flowchart of a process for diagnostic data mining of biological waves, in accordance with certain example embodiments;

FIG. 3 illustrates an example screenshot showing 6,465 hours of visually compressed signal (over 24000 heartbeats) recording of a 24 hours of Holter ECG, in accordance with certain example embodiments;

FIG. 4 illustrates a screenshot after decreasing the degree of visual compression at a region of recording that starts 64.97 minutes after the first beat in the recording shown in FIG. 3, according to certain example embodiments;

FIGS. 5A, 5B, and 5C illustrate morphologic analysis of beats taken at the onset of the pitch elevation, and at 12 and 67 seconds later, respectively;

FIG. 6 illustrates a screenshot taken 3.81 hours after the first beat in the region of recording shown in FIG. 3, in accordance with certain example embodiments;

FIGS. 7A and 7B show morphologic ECG display of beats at the onset and the peak of the pitch elevation, respectively;

FIG. 8 illustrates another schematic illustration of an example wave pattern visible with Spectral ECG, in accordance with certain example embodiments;

FIG. 9 illustrates an opening screen of a long duration SECG recording showing more than 50,000 heartbeats in the visually compressed mode, according to certain example embodiments;

FIG. 10 shows a less compressed part of the recording shown in FIG. 9, according to certain example embodiments;

FIG. 11 shows a discrete ST segment depression and a symmetrical and acuminated T wave—both indicators of myocardial ischemia, from the recording of FIGS. 9-10.

FIG. 12 illustrates a transition, in the recording of FIG. 9, from the ECG baseline to the left bundle branch block pattern, according to certain example embodiments.

FIG. 13 illustrates the same heart beats morphology as viewed using conventional ECG analysis techniques, and using SECG, according to certain example embodiments;

FIG. 14 is a color drawing corresponding to FIG. 3;

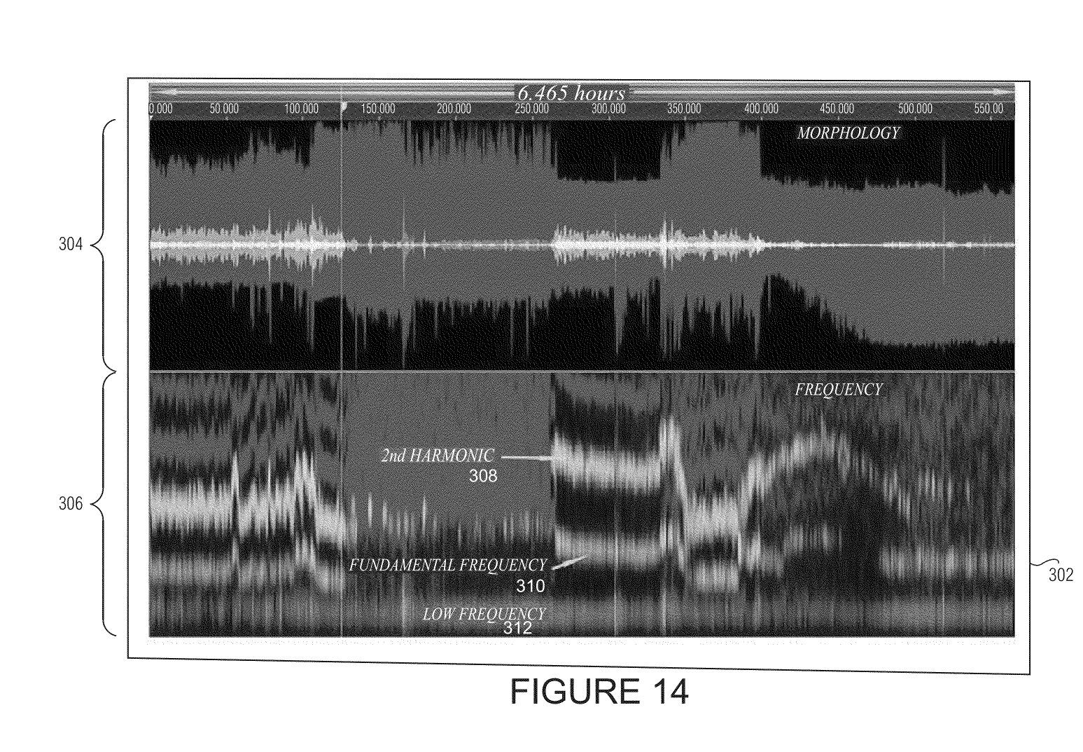

FIG. 15 is a color drawing corresponding to FIG. 4; and

FIG. 16 is a color drawings corresponding to FIG. 6.

### DETAILED DESCRIPTION

Certain example embodiments of the invention employ advanced digital and sound and other frequency analysis technologies to provide user-friendly cost-effective early risk identification of potentially lethal and/or catastrophic conditions in biomedical time series of any length. The biomedical time series may include, but is not limited to ECG, EEG, EMG, and the like.

According to an example application, embodiments are directed to finding the “hidden signals” suspected of being embedded within, for example, an ECG signal, as noted, for example, by Professor Goldberger in the article “Fractal Dynamics in Physiology: Alterations with Disease and Aging,” Proc. Natl. Acad. Sci. USA 2002 February 19; 99 (Supp 1):2466-2572. The article highlights the inadequacy of conventional ECG display and analysis techniques, which are suitable for stationary and linear non-biological signals, to detect certain hidden signal characteristics that may be critical to timely identification of dangerous biological conditions so that preventive measures can be commenced.

Certain example embodiments include methods and systems to record, preserve, retrieve, display, and/or analyze non-stationary, non-linear, biological waves with preservation of integrity, fidelity, and/or resolution of the biological waves using advanced sound and other modalities of frequency analysis technology. Risk identification, according to some embodiments, is based upon visual pattern recognition that may be learned and performed by physician-supervised trained technician. Certain example embodiments can be used in laboratory and/or clinical environments.

Some embodiments include a library of predetermined (e.g., previously identified) patterns used for analysis. The library may be made available locally at the computer where the analysis occurs or over a computer network (e.g., online). The library can be updated continuously or at regular intervals. Different types of pathology can be recognized quickly (e.g., at a glance) based upon characteristic patterns in the spectral frequency. The quick initial discrimination between normal and possible pathological ECG regions signs guiding the user to recording regions of diagnostic interest. Decompressed morphology and metrology analysis will render recognition of classical and novel ECG signs of cardiovascular risk that are greatly facilitated by changes in the spectral frequency display. According to some embodiments, the pattern recognition may be fully or partially automated. For example, in some embodiments, the system may automatically identify a portion of a recording that is similar to one or more previously identified patterns (e.g., from patterns stored in the library) and may alert the operator to more closely analyze that portion of the recording. The capabilities provided by certain example embodiments for rapid diagnosis makes them particularly suited to telemedicine and emergency services in addition to other laboratory and clinical environments (e.g., preventive cardiology, non-invasive diagnostic electrophysiology, intensive care settings, coronary care units, surgical suits, etc.).

When deployed in a clinical environment, for example, continuous updates to the patterns library, tutorials, and training regarding identification techniques, may be made available to users of the system. Additionally, a system that is centralized, Internet-based, global, 24-hour per day, staffed by cardiologists or other experts in the disclosed embodiments may be available to support users, including those serving telemedicine and emergency services.

The use of spectral sound analysis in certain example embodiments makes possible the early detection of pathological signals that are hidden and embedded within the conventional ECG, which cannot be detected using conventional digital ECG. Certain example embodiments introduce the novel capability to visualize the signals hidden within the traditional ECG signal, a capability not available in conventional ECG analysis techniques. Multiple color-coded singular layers may be used to isolate frequency ranges of interest. Selective gain sliders can be used to provide the user the capability to examine in detail the role of each frequency isolated in the constitution of the traditionally depicted ECG wave. According to some embodiments, the role of each isolated frequency in the morphologic signs of ischemic, arrhythmic waves, etc., can be fully examined.

The simultaneous color display of the wave morphology, time intervals, power, spectral frequency, pitch, and harmonics can also be displayed in a tridimensional rendition that facilitates the finding of events such as, for example, complex, dangerous, and/or premature ventricular beats that have peculiar characteristics that are recognizable with high certainty. By using multi-parametric simultaneous analysis, certain example embodiments can be supra-additive. In some embodiments, the totality of a long recording region can be displayed, visually-compressed, in one computer screen.

Since its introduction in clinical medicine more than 100 years ago, the ECG has been, and remains, limited to the time/voltage graphical rendition of the instantaneous mean electrical axes of all electrical forces simultaneously occurring in the myocardium all embedded within a thin single line. Electrocardiography interpretation is an acquired visual skill that relied on gestalt. Metrology, while contributing to documentation of blocks, voltages etc., is usually secondary to visual morphologic evaluation. Digital pattern recognition is not as good or can replace morphologic evaluation by a trained electrocardiogram reader. Thus, the inventors have, while enabling at least some embodiments to automatically perform digital pattern recognition, also enabled full or partial manual identification of such patterns by a person skilled in electrocardiography interpretation.

SECG is a novel method that separates and makes visible each individual current embedded and hidden within the instantaneous vector of the heart. For longer than half a century, it is known that myocardial electrical forces (other than the peak of the QRS) are in the 1 to 10 Hz range. SECG isolates frequencies of interest free of distorting contamination from noise and frequencies that are non-relevant. The frequencies of interest can be manually or automatically extracted to a separate layer. The layers can be color coded in the spectral and morphologic displays where it denotes their participation in the formation of the all inclusive conventional ECG. Single frequency layers can be merged with other chosen frequencies which may encode similar functional currents such as (but not limited to) the frequencies involved in the repolarization process of the myocardial cell. Abnormal repolarization reserve is a substrate that is predisposed to malignant arrhythmia. Frequencies between 1 to 10 Hz best represent atrial and ventricular repolarization.

SECG avoids contamination and distortion (by noise and frequencies of no immediate diagnostic interest) of currents of repolarization embedded and invisible in the conventional ECG. SECG facilitates quick and early disclosure of ECG signs of myocardial ischemia and abnormal repolarization reserve (e.g., congenital, disease and drug induced) among other conditions. In the visually compressed SECG mode, morphology and frequency spectrum patterns quickly guide a trained analyst to regions where baseline and pathologic events can be found.

Certain example embodiments provide for the quick and easy identification of artifacts in the biological signal, so that steps can be initiated to eliminate and/or otherwise address the cause of the identified artifacts, if considered necessary. The identification of the location in the frequency spectrum of electrical and other abnormal noise permits easy segregation of diagnostically-important aspects from the rest of the signal, thereby improving the speed, sensitivity and specificity of the analysis. Certain example embodiments provide for (potentially failsafe) alarms for arrhythmic, ischemic, bundle branch blocks, and other potentially dangerous transient events.

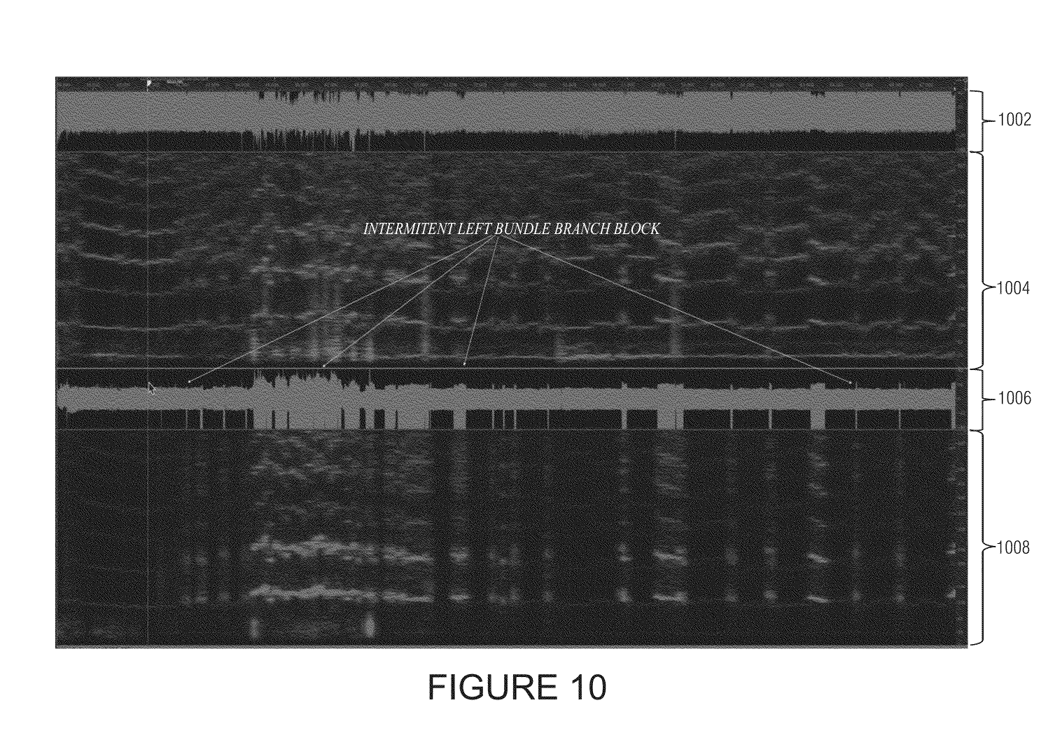

Atrial flutter and fibrillation, second and third degree atrio-ventricular bocks, bundle branch blocks, etc., have discrete compressed frequency patterns that allow identification of even very short silent transient events in the absence of symptoms or heart rate changes. That is possible even when the whole recording is first opened saving time in the diagnostic process that usually can be completed in single digit minutes (see, for example, description below relating to FIGS. 10-13).

A person skilled in the art would understand that certain example embodiments disclosed herein may be used advantageously in diagnostic situations that include rapid changes in visual compression in plural displayed characteristics of a biological signal, e.g., according to the best analytical strategy for a suspected diagnosis. Certain example embodiments may provide for using full or partial static screen capture to, for example, documenting the ECG signs that lead to the diagnostic interpretation. In some embodiments, motion screenshots can be used for archival purpose, research, training material, etc. Although ECG signals are used for description of many of the disclosed embodiments, a person of skill in the art will appreciate that the embodiments are not limited thereto.

Supervised, trained paramedical personal can visually inspect raw time series displayed in accordance with some embodiments, and quickly identify subtle and obvious differences in the structure of the data that may then lead to identification of the recording regions where signs of pathology are encoded. The diagnostic process can be completed within a few minutes after retrieval of long recordings obtained in consecutive hours or days. Diagnostic evaluation of SECG is also possible as the signal is being streamed in real time like in standardized stress testing, ambulances, intensive care and emergency hospital areas and telemedicine in general. Certain example embodiments disclosed herein make it unnecessary and undesirable to perform signal averaging for frequency spectral analysis after elision of abnormal beats as performed in conventional techniques. Indeed, such signal averaging eliminates signals encoded in abnormal beats in critical short-term conditions clearly abnormal, clearly evident in few signals surrounding the abnormal event evident in the original signal may be less evident, or not at all present, in the averaged signal.

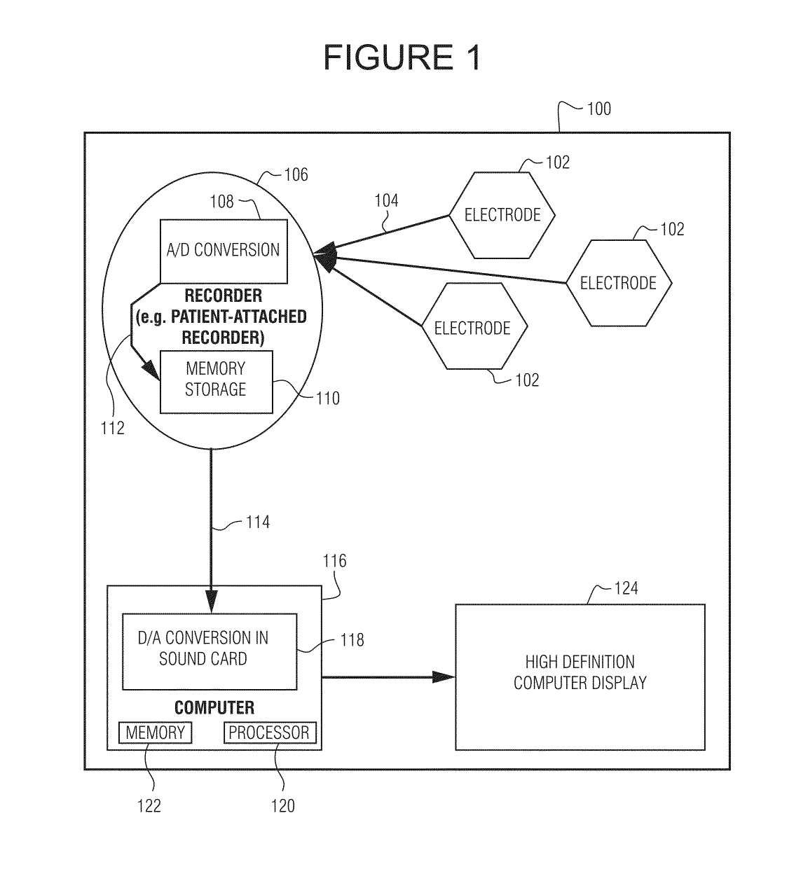

FIG. 1 illustrates a block diagram of a system 100 for diagnostic data mining of biological waves, in accordance with certain example embodiments. System 100 is configured for the recording, processing and analysis of biological signals.

According to an embodiment, a biological signal of interest (e.g., an ECG signal) is recorded with electrodes 102 or other appropriate transducers attached to the skin, scalp, and/or other area(s) of the body of a patient. Electrodes 102 may be X-ray transparent, non-magnetic, and/or may provide for amplification of the signal. Electrodes 102 may be communicatively connected via wired or wireless medium 104 to a recorder 10, which may, in some embodiments, be attached to the body of the patient (e.g., a patient-attached recorder). The biological signal (not shown in FIG. 1) received by electrodes 102 is processed by an analog to digital convertor (“A/D converter”) 108 in recorder 106. The digitized biological signal, according to some embodiments, is stored in a memory storage 110. Memory storage 110 may include a flash memory card or other type of non-volatile memory. In some embodiments, memory storage 110 includes a removable flash memory card. A/D converter 108 provides the digitized biological signal to memory 110 over a communication channel 112. Communication channel 112 may provide for transmission of control instructions in addition to data. Although in the illustrated embodiment, memory storage 110 is located in recorder 106, in other embodiments, the digitized biological signal can be transmitted to a device external to the recorder for storage, processing, and/or analysis without first storing the signal in memory 110. For example, communication channel 112 may include a network interface for communicating with an external device via a wired or wireless data transmission medium. Recorder 106 may include a processor (not shown) for controlling its operations.

According to some embodiments, the digitized biological signal is transferred from memory storage 110 to an analysis computer 116 over a communication channel 114 which includes a wired or wireless data transmission medium. In some embodiments where, for example, memory storage 110 includes a flash memory card in which the digitized biological signal is stored, the flash memory card is manually removed from recorder 106 and provided to computer 116 such that its stored content can be read. In the analysis computer 116, the digitized biological signal read from memory storage 110 is processed in a sound card 118 (and/or other processing resources such as, for example, a general purpose computer with at least one processor, a memory, etc.) that provides for digital to analog conversion (D/A conversion). The signal processed in sound card 118 or other device able to do spectral analysis. Spectral ECG, according to certain example embodiments, is a biological signal analysis program that can perform the spectral analysis from the sound card 118 or other device able to do spectral analysis. Spectral ECG may utilize some aspects of a program such as SpectraLayer™ to provide tools for waveform analysis. SpectraLayers™, created by Robin Lobel (listed as an inventor of this application) and marketed by SONY Corporation. SpectraLayers™, which is an advanced computer program designed for frequency spectral analysis, processing, and editing sound recordings in diverse media, is adapted to the analysis of long duration recordings of non-stationary, non-linear biological signals. Biological signals of interest include but are not limited to, the encephalogram, electromyography, retinal electrography, cardiac or any other biological sounds, electromechanical coupling of the heart, etc.

Morphological and spectral analysis of the signal is displayed in a high definition, high contrast display screen 124. Computer 116 includes a processor 120 and memory 122. Processor 120 provides for controlling operations in computer 116 and executes biological signal analysis programs. Processor 102 may include a central processing unit (CPU) and/or one or more specialized processors (e.g., a math co-processor, a graphics co-processor, a digital signal processor, ASICs, etc.). Memory 122 may include volatile and/or non-volatile memory and may provide for storing programs and data elements such as the biological signal analysis programs executed by processor 120, configuration parameters for the biological signal analysis programs and/or biological signal acquisition, received biological signals, and/or a library of predetermined signal patterns.

Processor 120 executes a biological signal analysis program using the biological signal processed in soundcard 118 or a similar frequency analysis device. The biological signal, or aspects thereof, is displayed on the screen 124. The program may be configured to provide a plurality of controls (e.g., sliders, buttons, scroll bars, etc.) to adjust contrast, luminance, independent gain controls for the frequencies isolated as well as for the morphological display, etc. The biological signal analysis program may provide for the user to choose different levels of signal compression to visualize extended periods (e.g., multiple hours) of recording or single beats, including parts thereof, in the full screen using improved or optimum configurations for each chosen mode.

Long electrocardiographic Holter recordings can be processed by computer 116 according to the methods and techniques described in U.S. Pat. No. 6,370,423 (the '423 patent), which lists Juan Guerrero and Christian Guerrero as inventors. The content of the '423 patent is incorporated herein by reference in its entirety.

The '423 patent improves digital extraction of a biological signal, such as the analog ECG, with total integrity, ultra high fidelity and resolution for optimum (or nearly optimum) signal-to-noise ratio. The techniques disclosed in the '423 patent also avoid signal distorting or destructive steps in digital electrocardiography in conventional systems. The techniques enable greater accuracy of morphologic and time intervals evaluation than conventional digital ECG methods.

While the use of sound-derived technology, as described in the '423 patent may require less, or shorter, training for users than for conventional analog ECG. The techniques described in '423 may be further improved through sound or other frequency analysis technology able to discriminate the signal embedded with the averaged representation of the signal of interest the goal are methods that are fast, cost-effective, user-friendly, and fast for use by supervised paramedical staff in telemedicine, intensive care, emergency situations, and/or the like.

After a quick inspection of the multiple ECG leads recorded, one lead (channel), for example, the one with the best signal quality, can be chosen for the analysis of hundreds or thousands of recorded heart beats. The morphology and spectral analysis panels open horizontally aligned in exact temporal synchronicity. Diagnostic data mining starts with examination of the fundamental frequency pitch looking for the lowest and highest pitch in the recording region displayed. The lowest pitch usually corresponds to the most basal (as normal as it will be) condition for a given recording in a patient. The highest pitch, especially if concomitant with onset of unusual frequencies or other signs, usually leads to the area where most pathology is likely to be encountered. Signal data obtained at basal conditions (usually when the patient is sleeping) is the best frame of reference to evaluate pathological waves encountered in the same recording.

The use of spectral ECG for digital data mining of biological waves in some embodiments provide supra-additive combined analysis capabilities. Spectral ECG allows quick identification of the baseline (as normal a signal is within a patient recording) as well as recording regions where pathologic signs may be encoded. The baseline is characterized by the lowest pitch (in Hertz) that often corresponds to the color luminance and hue that characterizes low power within the signal. Concomitantly, abnormal frequency bands in certain ranges of interest are absent. Pathology within a recording region is often marked by a gradual—often rapid—onset and offset elevation of the pitch. Another very important sign is the sudden onset of abnormal frequencies in areas of interest. If abnormal frequencies were present, the signal in the frequency of interest becomes continuous, and of higher luminance often changing in hue and configuration.

Supervised, trained paramedical personal can visually inspect raw time series displayed in SpectraLayers™ and quickly identify subtle and obvious differences in the structure of the data that lead to identification of the recording regions where signs of pathology are encoded. Data mining with SECG makes possible to complete the diagnostic process within few minutes after retrieval of long recordings obtained in consecutive hours or days. The supervising physician can elect to further identify signs of pathology with the assistance of a patterns library, expert advice through the interne, or send the recording and the patient for further evaluation at a cardiology unit.

After a quick inspection of the multiple ECG leads recorded, one lead (channel) (e.g., the one with the best signal quality) can be chosen for analysis of hundreds or thousands heart beats recorded. The morphology and spectral analysis panels open horizontally aligned in exact temporal synchronicity. Diagnostic data mining may be started by examining the fundamental frequency pitch looking for the lowest and highest pitch in the recording region displayed. The lowest pitch usually corresponds to the most basal condition for a given recording in a patient. The highest pitch, especially if concomitant with onset of unusual frequencies or other signs, usually leads to the area where most pathology is likely to be encountered.

Preceding the peak of a transient ischemic event, the power of the ECG signal usually increases. Power changes are best seen in the frequency display channel in luminosity and hue than in the corresponding dimensions in the voltage domain of the morphologic panel. It is also common to see abnormal frequency bands, likely to represent abnormal myocardial cell repolarization, that may appear de novo and that change in luminance or tonality as the ischemia, or other pathology progress. Depending on the degree of pathology in a given patient, the abnormal frequencies can be permanent; if so, power (dB, micro volts) increases as the ischemia becomes more severe during an episode of transient ischemia or arrhythmia aggravation. The range of the abnormal frequency can also become wider. Other frequency ranges may also be present or appear de novo according to the pathologic condition of the patient. The power within the frequency bands is likely to wax and wane according to ischemia or arrhythmia severity. Differences between the 1^{st} and 2^{nd} Harmonics are likely to identify pro-arrhythmic risk, probably because of myocardial structural abnormalities. The observation of these different, simultaneous, pathophysiologic changes assures the specificity of the findings.

In certain example embodiments, proper settings allow single beat morphology and frequency analysis to permit precise time identification of the beginning and end of each component of each ECG wave and interval. The precise location of markers becomes possible ending current uncertainty regarding crucial marking placement for exact analysis of time intervals. When morphology alone is used in conventional ECG analysis today, there are important points of diagnostic interest (e.g., exact location of the J point, true end of the T wave etc.) that can be obscured by current digital poor resolution and fidelity or the pathology-induced changes in the waves morphology. The synchronic, simultaneous, morphologic, temporal and frequency analysis, which can be magnified (e.g., zoomed-in/out) at will, resolves issues with respect to such uncertainty.

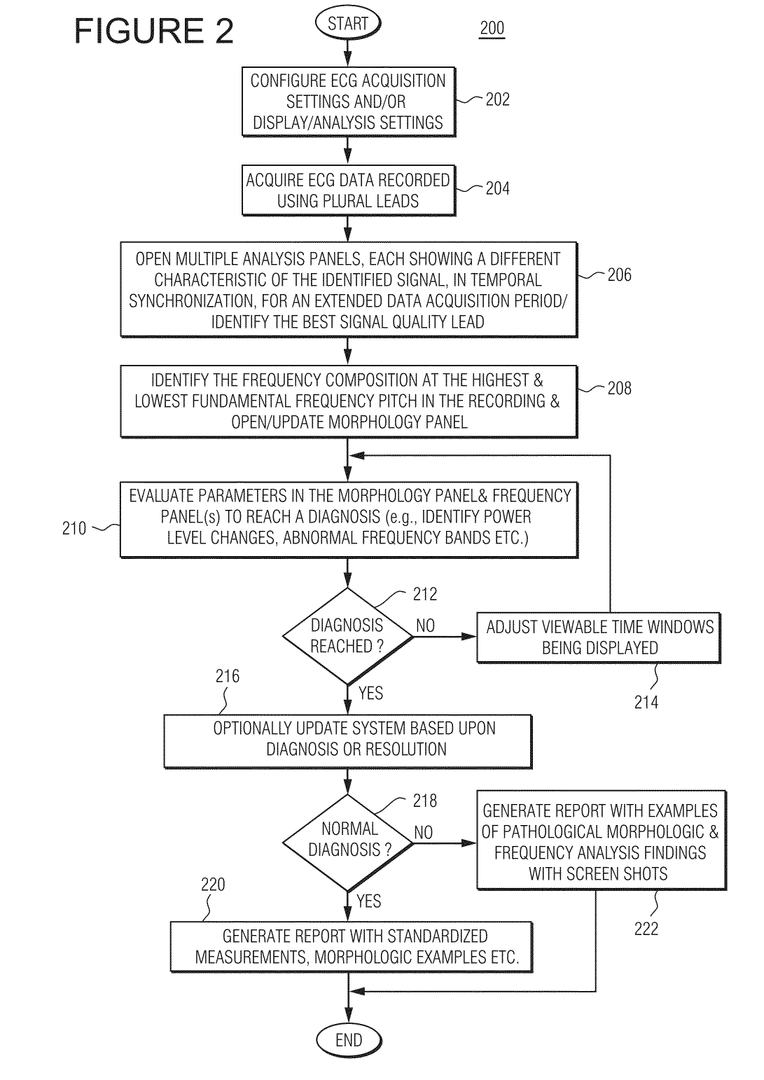

FIG. 2 illustrates a flowchart of a process for diagnostic data mining of biological waves, in accordance with certain example embodiments.

At operation 202, the biological signal acquisition system and/or analysis and display system is configured. The configuration of the biological signal acquisition system may include electrode configurations (e.g., number and placement of electrodes, signal amplification etc), and acquisition parameters (e.g., ECG acquisition parameters) such as power levels, length of acquisition, and the like. The configurations may also include A/D conversion parameters, storage parameters (e.g., location for storing acquired signal information).

The configuration of the analysis and display system may include, but is not limited to, selection of a number of panels to display simultaneously and synchronized in time. The recording region to be analyzed, visual compression settings, etc. can be configured. The configuration may also include configuration of the settings for all the sensing devices parameters. Special transducers can be added to the ECG electrodes for continuous recording of oxymetry, blood pressure, cardiac and carotid sounds, respiratory functions etc. In different configurations, ECG, EEG, respiratory function signals, etc., may be simultaneously acquired to better understand the relationships between the heart, brain and respiration functions in epilepsy, schizophrenia, sleep apnea, heart failure, syncope, etc. The recording device has a gyroscope to keep track of the patient's body position eliminating that as a possible confounding diagnostic factor.

At operation 204, the biological signal from the patient is obtained in accordance with the configurations. In certain example embodiments, the biological signal obtained is an ECG signal obtained from one or more electrodes placed at selected locations on the patient's body. As described in relation to FIG. 1, the obtained signal may be processed by an A/D converter and stored in a memory. On the analysis computer, the digitized biological signal may be processed using a soundcard as, for example, described in the '423 patent. Using a sound card enables the digitization of substantially greater frequency ranges than that achievable in current ECG chips (e.g., 192,000 Hz in sound card compared to 10,000 Hz in conventional ECG chips). Moreover, sound cards offer better signal-to-noise ratios, wider dynamic range, achieve ultra high fidelity and definition of the signal recovered. The processed biological signal may then be displayed and/or analyzed using the biological display and/or analysis program including a sound analysis program such as, for example, SpectraLayers™.

At operation 206, a plurality of analysis panels is displayed, with each panel showing a different aspect of the biological signal. The panels may display an extended temporal period, such as several minutes, one or more hours, one or more days, or even longer durations. The recording period initially displayed may be configurable. According to an embodiment, for example, the visually compressed ECG signal for 24 hours is provided as the initial display. Each panel displays its content in exact time synchronization to the content displayed in the other panels.

The biological signal from one electrode may be selected for display. In other embodiments, the signal acquired from sensors other than ECG electrodes (e.g., oximetry, EEG electrodes, microphones, etc.) may be displayed.

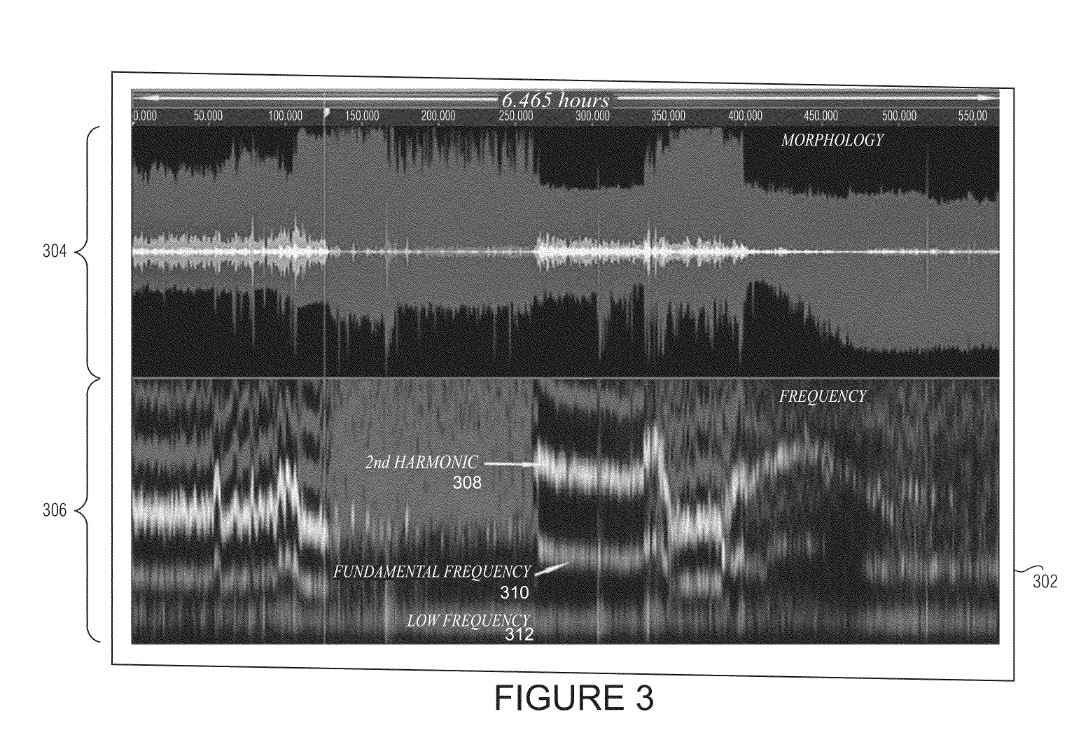

At operation 208, the content of the displayed panels are visually examined to determine frequency composition at the highest and lowest fundamental frequency pitch in the recording. The fundamental frequency shown in a display according to an embodiment is shown in FIG. 3. In some embodiments, the highest and lowest pitches may be automatically identified. In some embodiments, a morphology panel is opened. In some other embodiments, the morphology is displayed in an already open panel.

At operation 210, the displayed multiple panels, for example, the morphology and frequency panels, are evaluated for their parameters. For example, power level changes, and abnormal frequency bands are identified. According to an embodiment, the identification is performed visually. According to another embodiment, the identification is automatically performed.

At operation 212, it is determined whether a diagnosis of normal or abnormal is reached. Diagnosis may include the observation, visually by the user, of the different characteristics of the biological signal displayed in separate panels (e.g. morphological and frequency panels) to make a determination that a particular biological condition is indicated by the biological signal. The visual inspection may be further informed by a library of predetermined patterns that may be accessible to the user. In some embodiments, predetermined patterns in the library may be automatically matched to the biological signal being analyzed, and the user may be prompted to areas where substantial similarity between a predetermined pattern and the signal being analyzed is found.

If a diagnosis is reached, then at operation 216, the system is optionally updated based upon the results of the diagnosis. For example, the library of predetermined patterns may be updated to include the biological signal characteristic and pattern in association with the diagnosis. Following operation 216, at operation 218, it is determined by the operator whether the recording shows normal or pathological waves.

If the diagnosis is a normal diagnosis, then at operation 220, processing for normal diagnosis may take place. The processing may include preparing a report with standardized measurement, morphological examples etc. A comment may be entered by a responsible physician in a generated report for the file.

If the diagnosis is abnormal, then at operation 222, processing for abnormal diagnosis may take place. Processing for abnormal diagnosis may include documenting examples of pathologic morphologic and frequency analysis findings, for example, with static or dynamic screen shots. A comment by a responsible physician in a generated report may include a list of possible differential diagnosis. After either 220 or 222, process 200 may end.

If the diagnosis of normal or abnormal is not arrived at earlier, then at operation 214, the displayed time window is adjusted. The adjusting of the displayed time window may be performed such that the user can zoom-in to particular areas of the displayed biological signal. In effect, the visual compression of the displayed portion of the signal is changed so that the user can focus into an area of the signal that includes an abnormality of interest. The user may control the level of visual compression, magnification, contrast, bi- or tri-dimensionality etc., of what is displayed, and the portions of the signal to be further examined.

Operations 212-222 may be repeated by the user in order to repeatedly display, at different levels of visual compression, an area of abnormality and portions of the signal to either side of the abnormality area. After one of more occurrences of operations 212-222, the user may successfully complete the diagnosis and/or resolution of an abnormality seen initially at a high level of visual compression of the biological signal being analyzed.

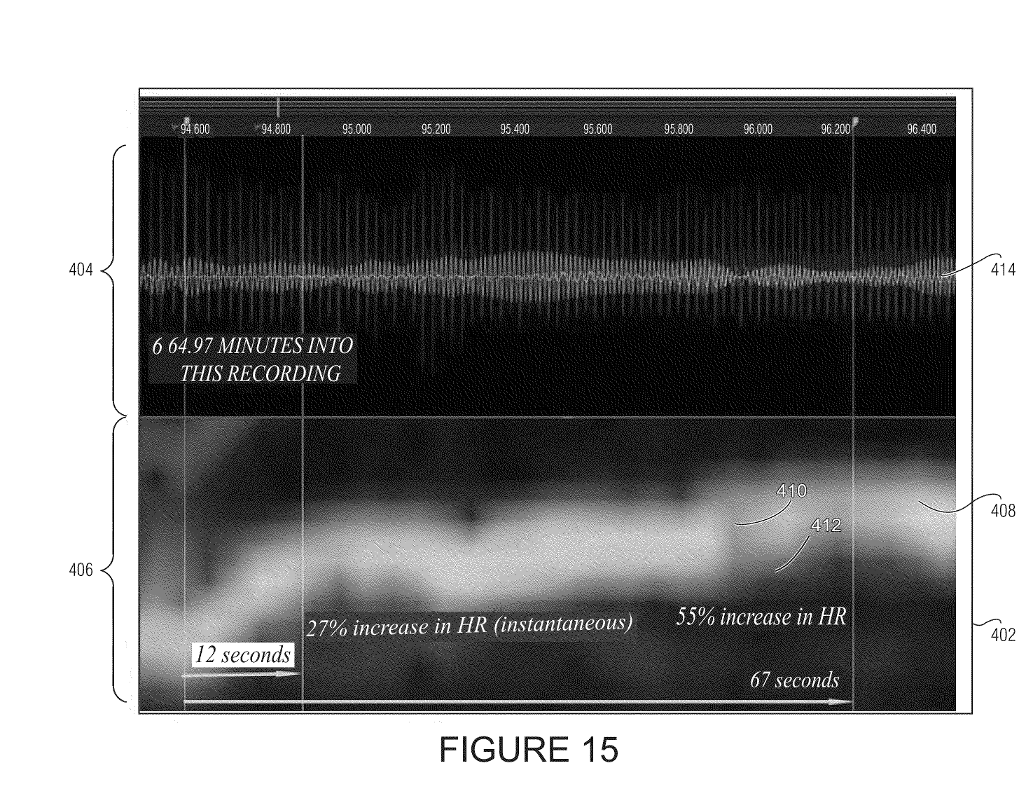

FIG. 3 (also corresponding color drawing FIG. 15) illustrates an example screenshot showing the signal for part of a 24 hours recording of a Holter ECG, in accordance with certain example embodiments. The analog ECG signal was digitally extracted from magnetic tape recordings using the method described in the '423 patent. The screenshot corresponds to a period of 6.465 hours that includes about 25,000 heart beat ECG complexes. To avoid crowding the screen 302, one lead was selected for display. The upper panel 304 shows the conventional morphologic ECG signal visually compressed in the time domain (e.g., visually compressed along the x-axis). In the upper panel, the y-axis represents power variations. The lower panel 306 shows the frequency spectrum display in exact synchrony in time with the morphologic display. The different color bands (shown in color in FIG. 15 and shown in respective grayscale in FIG. 3) show selected frequencies isolated.

To a trained user, it becomes immediately apparent that this ECG signal is not from a normal (or healthy) subject. The on and off presence of the fundamental frequency 308 (shown in violet in FIG. 15) and other frequencies and harmonics (shown in green in FIG. 15) is considered to be highly correlated with intermittent atrial fibrillation. The pattern of the low frequency 312 (shown in purple in FIG. 15) at the bottom of the panel is suggestive of abnormal ventricular muscle repolarization reserve.

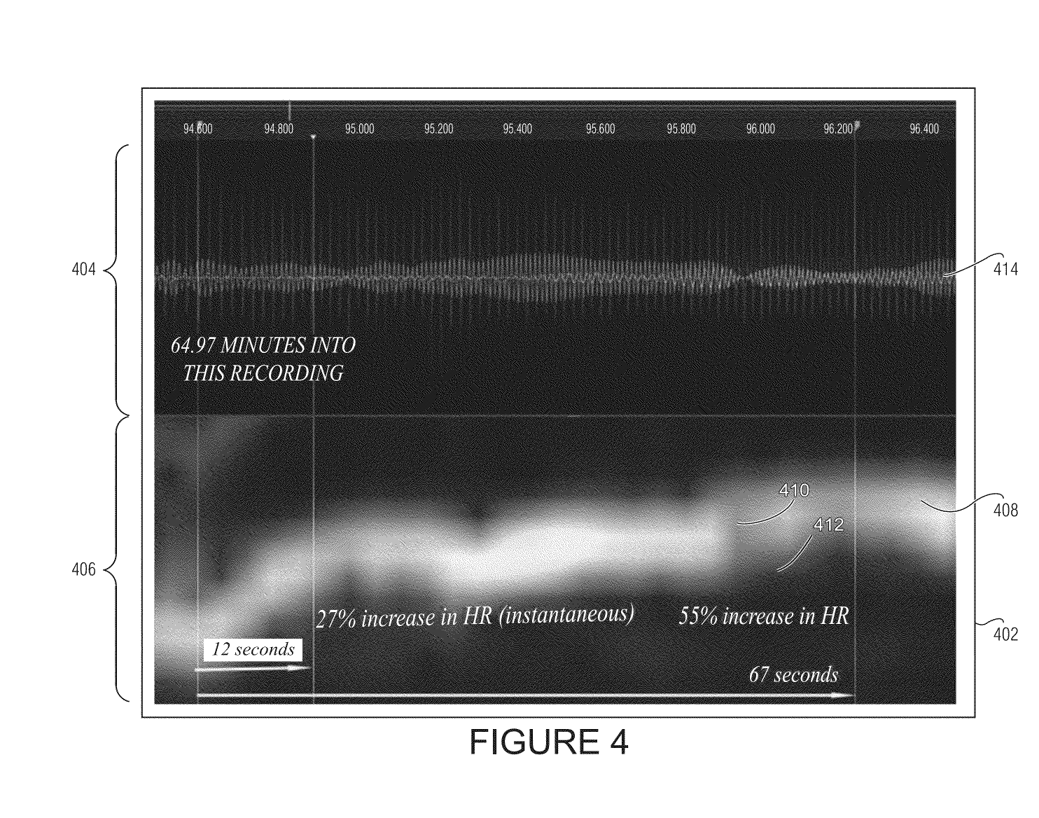

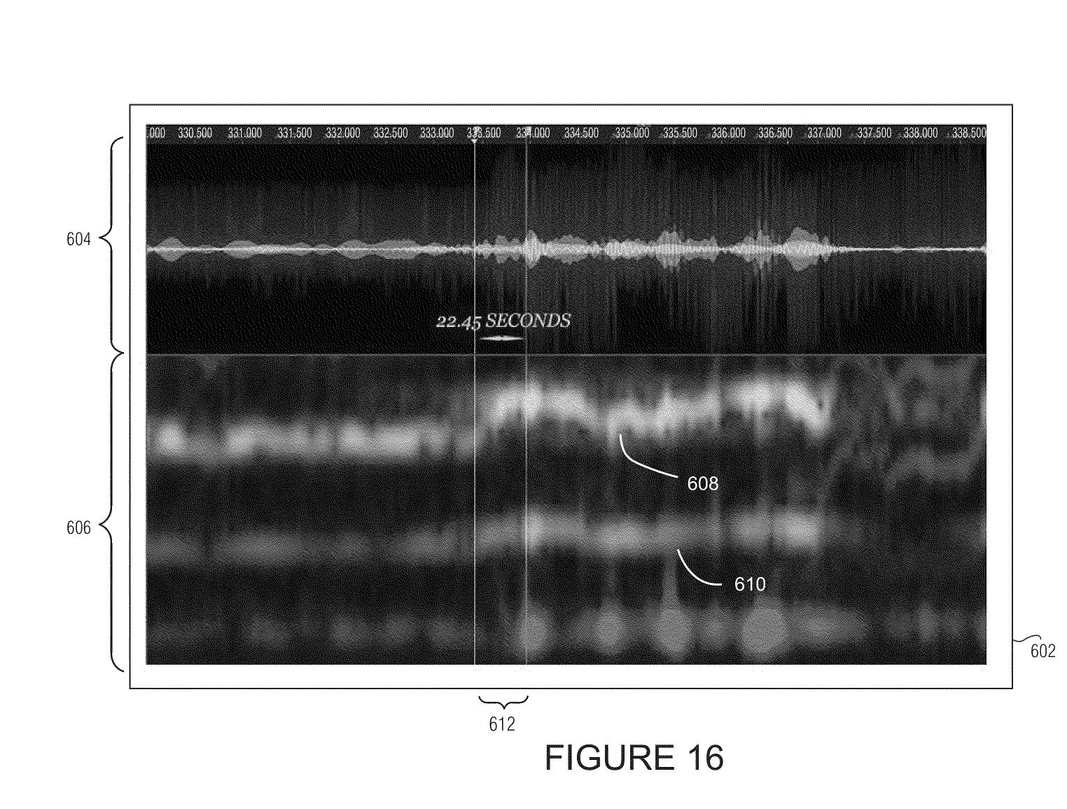

FIG. 4 (also corresponding color drawing FIG. 16) illustrates a screenshot after decreasing the degree of visual compression at a recording period that starts 64.97 minutes after the first beat in the recording shown in FIG. 3, according to certain example embodiments. For this example, the 2nd Harmonic 408 (e.g., a portion of the 2nd Harmonic 310 in FIG. 3) was chosen for display on the screen 402. Variations in grayscale at 410 and 412, for example, within the band 408 represent power changes within a frequency range (see also the green, red, and yellow visible in the corresponding color drawing FIG. 16). In the conventional ECG morphology displayed compacted in panel 404, the power changes within this frequency are shown by the variations in height of the green sinusoid line 414 (shown in green in panel 904 in corresponding FIG. 16). It is noted that independent power gain for this frequency range was used to improve visualization of the power fluctuations. A significant elevation in the pitch is clearly visible in panel 406 reaching its maximum level within 12 seconds of the pitch elevation onset. To the trained user, such elevation in the pitch is a clear sign to focus attention to an area of possible diagnostic interest in the recording. Twelve seconds after the onset of the pitch elevation, the instantaneous heart rate had increased by 27% (from 49 to 62 beats per minute) to reach a maximum increase of 55% (from 49 to 76 beats per minute) 67 seconds after the onset of the pitch elevation. It is to be noted that these changes in the heart rate are within the normal range therefore conventional digital ECG event record may not be saved the signal (e.g. may be deleted and/or written over), the diagnosis would be missed.

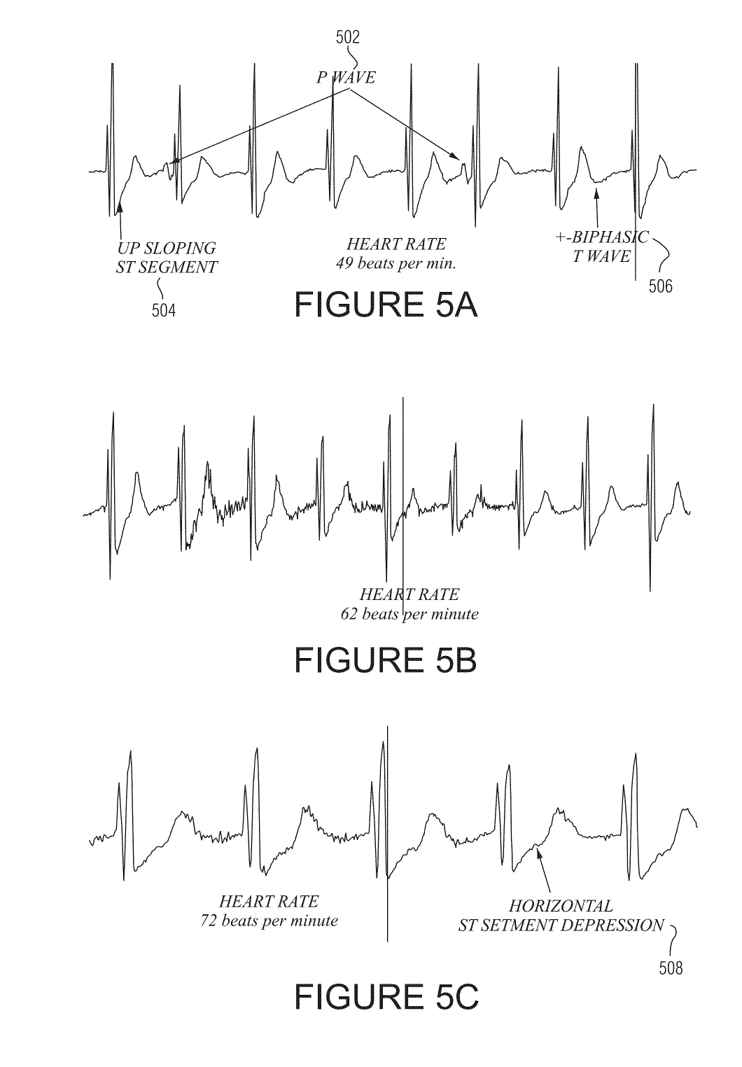

FIGS. 5A, 5B and 5C illustrate morphologic analysis of beats taken at the onset of the pitch elevation, and at 12 and 67 seconds later, respectively. FIG. 5A shows beats at the onset of the pitch elevation episode. There is a QRS pattern indicative of right bundle branch block that is a usual exclusion for a traditional exercise stress test. The up-sloping ST segment 504 elevation present is wrongly considered a normal variation. Classic fibrillatory waves are not seen, and the occasional P waves 502 are indicative of intermittent absence of atrial fibrillation. It is important to pay attention to the slower than normal descending limb of the T wave 506 with a terminal negative deflection that suggest elongation of the T peak-to-end period with a +/−Biphasic T wave pattern, both signs of possible ischemia and/or another cause of abnormal ventricular repolarization reserve. FIG. 5B, taken 12 seconds into the pitch elevation episode, shows absence of the P wave, and the atrial fibrillation waves are evident and constant at this time. It is important to note that the T wave has peaked and become acuminated, with marked variation in the voltage (y-axes) domain, which fit the description of T wave heterogeneity; an ECG sign of ischemia and/or abnormal ventricular repolarization reserve. FIG. 5C is taken 67 seconds after the baseline panel, with a lower degree of visual compression than the two above. At this time the heart rate was 55% over baseline, and still within the normal range, and hence not preserved by conventional ECG event recorders. This panel shows classical horizontal ST segment depression 508 and T peak to end elongation, both signs of ischemia and abnormal ventricular repolarization reserve.

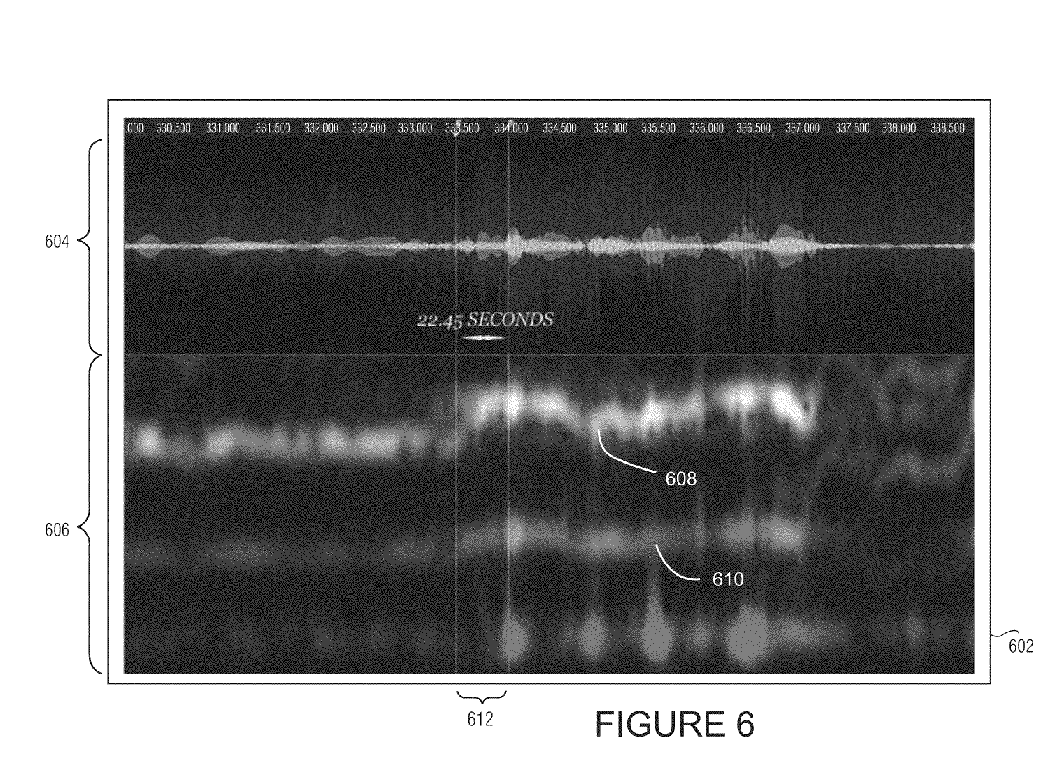

FIG. 6 (also corresponding color drawing FIG. 17) is a screenshot illustrating a time period 3.81 hours after the first beat in the recording region shown in FIG. 3, in accordance with certain example embodiments. The screen 602 includes panels 604 for morphology and panel 606 for frequency. The 2^{nd} Harmonic 608 and the fundamental frequency 610 are both illustrated in the lower panel 606. FIG. 6 shows another period 612 of 2^{nd} Harmonic 608 (shown in green in FIG. 17) pitch elevation, which can also be seen, although less clearly, in the fundamental frequency 610. The changes in power of the signal at the different frequency ranges are shown as changes in color (shown in FIG. 6 as changes in grayscale) and luminance in the spectral frequency bands. In this period, the pitch elevation peak was reached 22.45 seconds after the onset of the elevation.

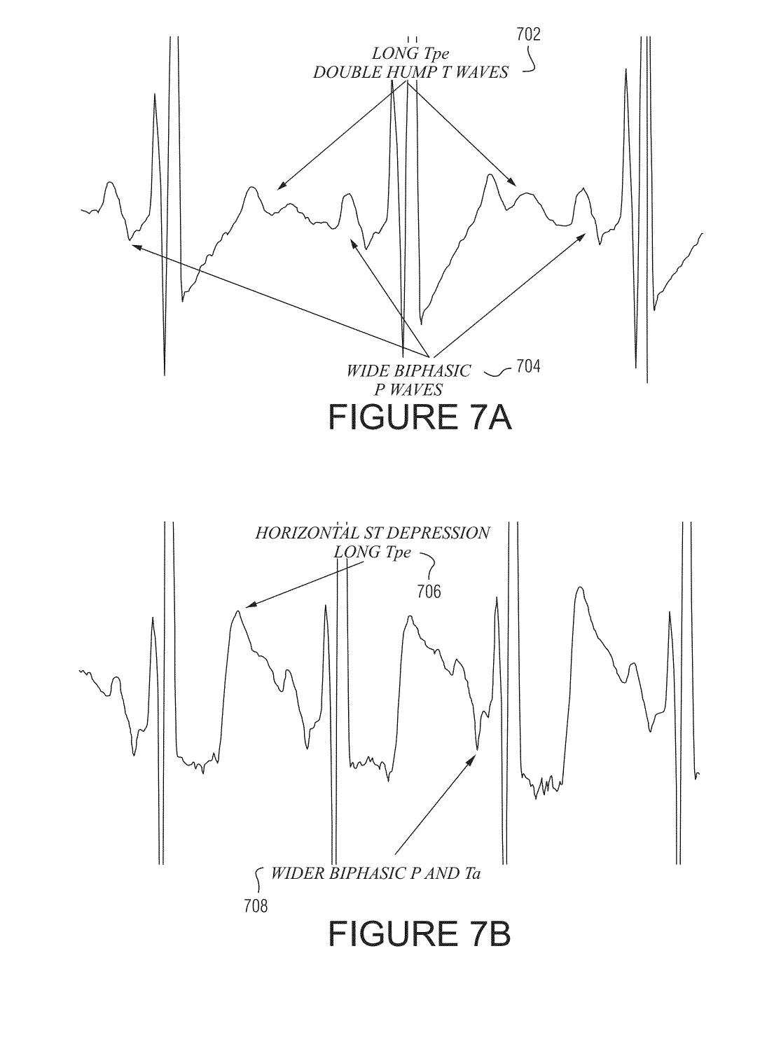

FIGS. 7A and 7B show morphologic ECG display of beats at the onset and the peak of the pitch elevation, respectively. At this time, there is normal sinus rhythm, P waves 704 precede the QRS. The P waves are more biphasic and wider (longer in duration) than those in FIGS. 5A-C suggesting atrial overload a frequent cause of atrial fibrillation often due to arterial hypertension. The instantaneous heart rate at the onset was 70 beats per minute; and 82 beats per minute at the peak of the event, still within the normal frequency range. It is important to note that the PQ interval, which represents atrial repolarization, is markedly depressed 22.45 seconds after the onset of the mild (and within the normal range) heart rate elevation. This ECG sign 708 is known as the Ta, a neglected and forgotten marker of atrial muscle ischemia that is not seen in current digital electrocardiogram. At the onset of the pitch elevation, the ST segment was still up-sloping which often is, taken out of context of further developments in the recording, wrongly considered a normal variation. At the peak of the pitch elevation a classic horizontal ST segment depression 706 indicative of ventricular ischemia is observed. The T peak to end is still elongated. In summary, pitch elevation is a good example of features of certain example embodiments, which by visual compaction of scores of thousands of heart beats displayed in the traditional morphologic and frequency display modalities quickly guide the user to areas of the recording where ECG signals of pathology can be readily found that are currently routinely missed by conventional digital ECG technology. FIG. 7A also illustrates Long Tₚₑ and double hump T waves 702. It is known for more than a decade that the morphology of the terminal end of the T wave—acumination, double or triple humps etc—is of great importance to diagnose abnormal ventricular repolarization reserve congenital, acquired or drug-induced in origin. These are clear signs of risks for potentially lethal ventricular arrhythmia. Conventional ECG techniques lack the resolution and fidelity to make these signs visible, and therefore dangerous false negative reports in these regard are the norm. That is, the false negatives are not due to the signals not being present, but to the inability of the conventional techniques to identify and/or make evident such signals. (See, e.g., J. Willis Hurst, M. D., “Current status of Clinical Electrocardiography with suggestions for the improvement of the interpretative process,” The American Journal of Cardiology, Vol 92, pp. 1072-1079, November 2003; C. M. Yong et al., “The Electrocardiogram at a Crossroads,” Circulation 2013, 128:7982; N. A. Mark Estes III., “Computerized Interpretation of ECGs, Supplement Not a Substitute,” Circ. Arrhythm. Electrophysiol., 6:2-4, 2013.

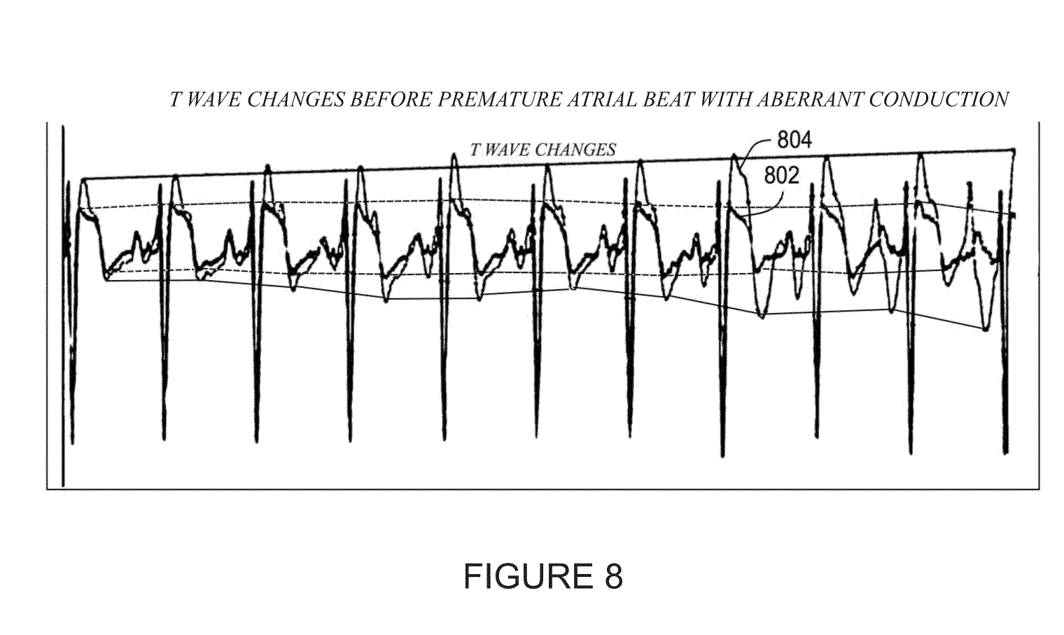

FIG. 8 provides another schematic illustration of signal patterns visible using conventional ECG and SECG, from the same recording. Signal pattern 802 shows the upper and lower bounds of the T-wave according to the conventional ECG. Signal 804 shows the T-wave upper and lower bounds as shown by SECG. The progressive growth in the height of the T-wave—a sign of pathological increased power—shown by 804 is a sign of abnormal ventricular repolarization which conventional ECG fails to detect. Signal pattern 804 reveals a problematic ventricular faulty repolarization reserve (which may, for example, be due to genetics, disease, or drugs) that increases the risk for arrhythmia. Conventional digital ECG with frequency signal averaging or any other form of mathematical abuse of non-stationary, non-linear biological signals, cannot uncover this condition from the ECG signal.

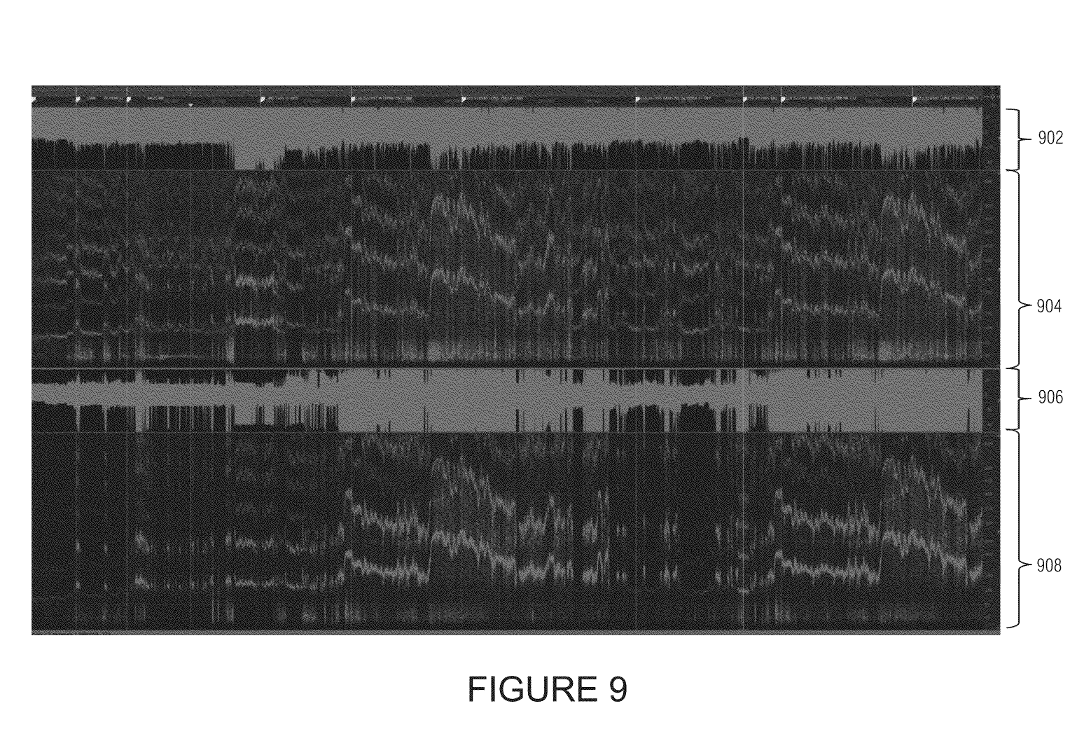

FIG. 9 is the opening screen of an 11 hours and 20 minutes SECG recording showing more than 50,000 heartbeats in the visually compressed mode. The 902 and 906 panels are the compressed morphology/time display of two different lead recordings (by design these are different electrodes placed on different parts of the pectoral area to capture the electric signal). It is clear that the frequency/time spectrum shown in the 904 and 908 panels are not similar. This visual pattern immediately suggests the diagnosis of intermittent bundle branch block.

FIG. 10 shows a less compressed part of the recording shown in FIG. 9. Very short, intermediate, and long duration periods of left bundle branch block can be recognized (marked in the drawing) and are clearly seen in panel 1008. This serious electrical conduction abnormality can be asymptomatic and a prelude to sudden cardiac death. It generally requires emergency management of the patient by a cardiologist.

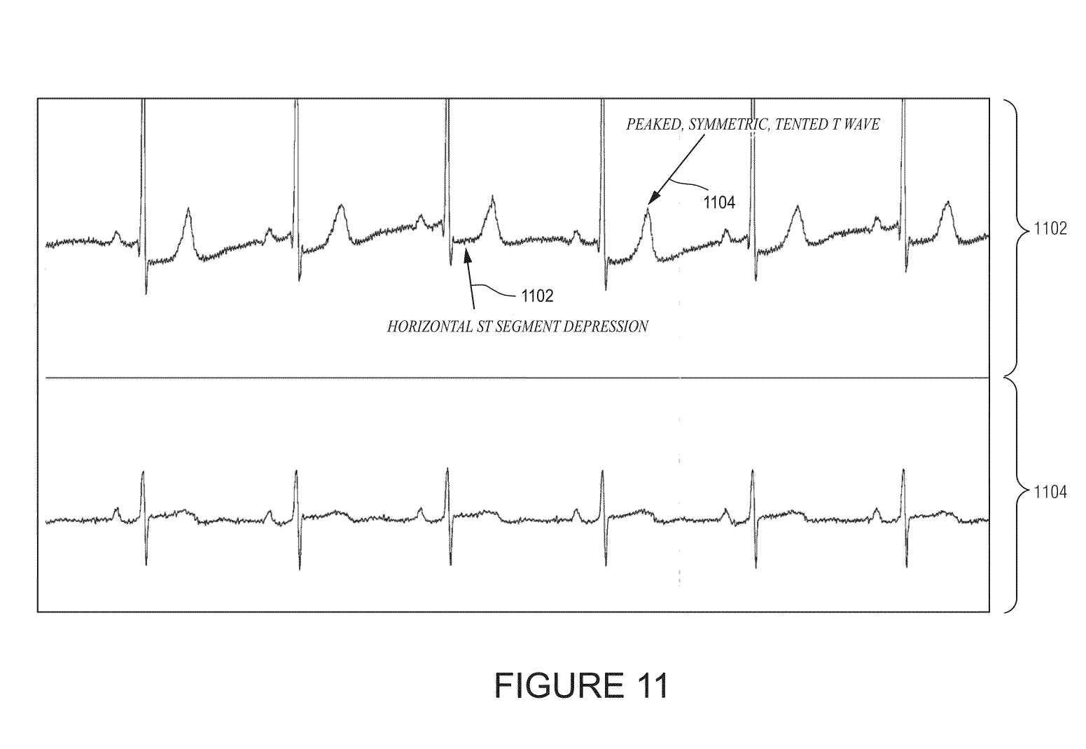

FIG. 11, taken from the same recording as FIG. 9 in a further decompressed mode of panels 902 and 906, shows a discrete ST segment depression 1102 and a symmetrical and acuminated T wave 1104 of normal height—both indicators of myocardial ischemia. The conventional digital ECG lacks the necessary resolution to visualize these discrete changes. SECG has superior resolution and fidelity and allows magnification of the recording to thoroughly examine the SECG signal displayed.

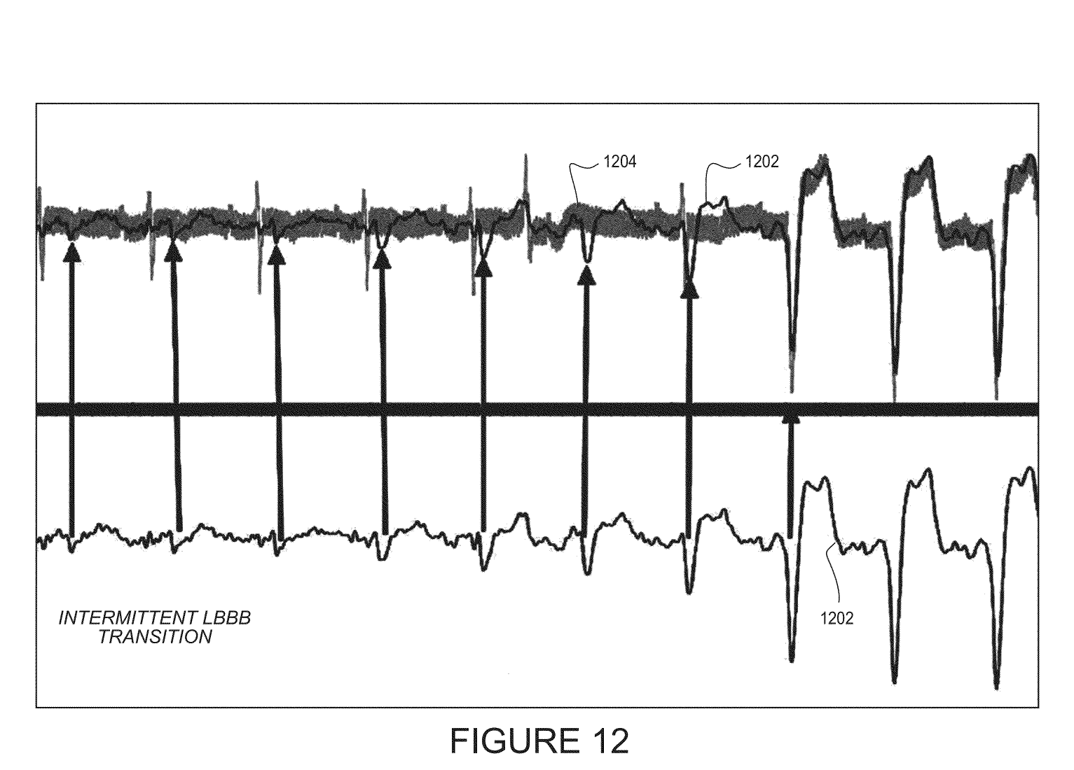

FIG. 12 shows the transition, in the recording of FIG. 9, from the ECG baseline to the left bundle branch block pattern, which is clearly visible despite the noise. The SECG signal 1202 is the summation of two repolarization frequencies, without the noise or frequencies that are of no interest, to best depict pathological ventricular repolarization compatible with left bundle branch block.

FIG. 12 shows the transition a very noisy traditional ECG in the upper panel with the embedded frequency of interest 1202. The lower panel is the SECG frequency of interest 1202 isolated without the noisy conventional ECG 1204 in the upper panel. It is to be noted (arrows) that a progressive widening of the QRS starts (indicative of ventricular conduction delay) and elevated ST segment and T wave (abnormal ventricular repolarization) are shown by SECG several beats before they become manifest in the conventional ECG. The impression obtained from the opening screen (FIG. 9) is confirmed; the patient needs prompt specialized attention.

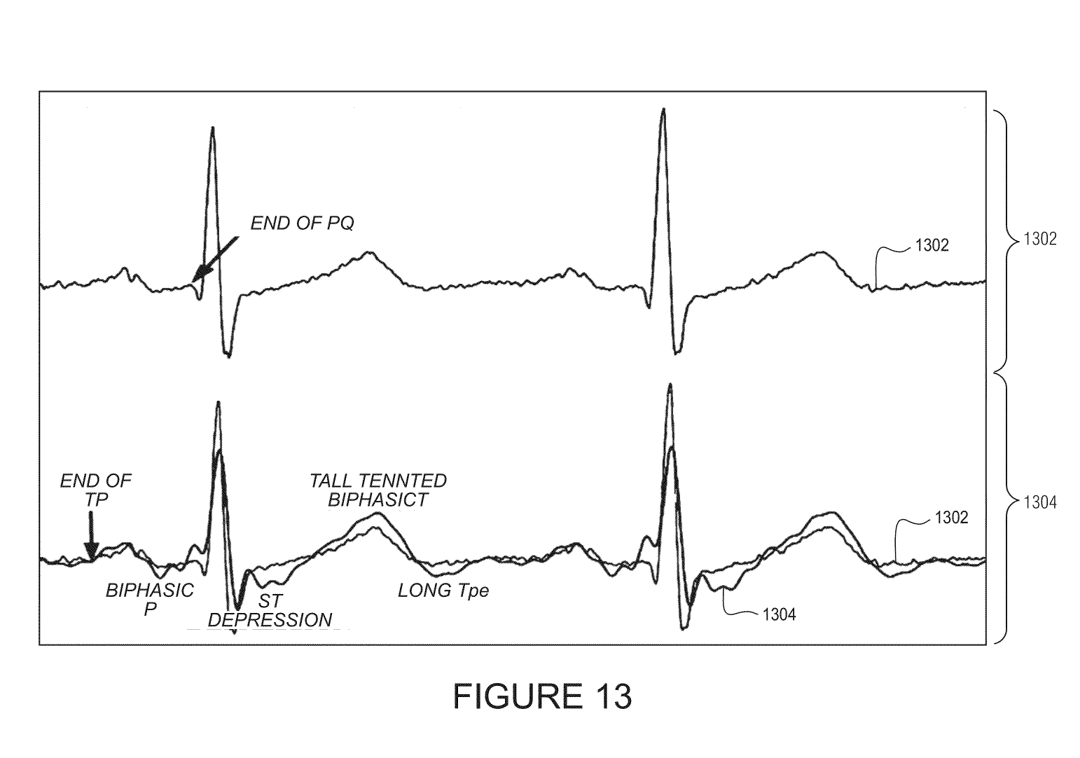

FIG. 13 shows the same heart beats morphology in both panels., the upper in the conventional ECG as per the 1998 patent, the lower with the SECG frequency on interest morphology. The magnetic tapes from which the digital extractions were done came anonymously and without any clinical information. Under those circumstances this patient recording was labeled as quasi normal when all we had was the morphology 1302, in the lower panel. SECG has made possible signal 1304 which illustrates the isolation of biphasic P wave and horizontal ST segment depression, Biphasic tented T morphology with elongation of the T peak to end indicative of decreased repolarization reserve. This condition could be congenital, or secondary to multiple diseases (mainly cardiomyopathies) or drugs. Decreased ventricular reserve is the substrate for life compromising or disabling arrhythmia.

Certain example embodiments described above provide for a novel technique for diagnostic digital data mining of non-stationary, non-linear, biological waves to permit novel, cost-effective, user-friendly, rapid and early diagnosis (e.g., sufficiently early to forestall further pathologic progression) of the risk for potentially lethal or catastrophic conditions timely enough to institute preventive therapy. As noted above, the non-linear, non-stationary characteristics of biological waves are unsuitable for effective diagnostic use of digital analysis of biological signals as done today in conventional systems. Certain example embodiments enable multiple characteristics of the biological waves registered in long recordings lasting minutes, days, or weeks, can be displayed simultaneously, in one computer screen, at different levels of visual compression, e.g., to help identify significant changes in each parameter to be evaluated within the context of all the other signal characteristics with supra-additive diagnostic power for pathophysiological interpretation of complex fluctuations of non-linear, non-stationary biological signals.

Certain example embodiments may include processing and/or analysis of short- and/or long-term recordings of biological signals such as, for example, the ECG, EEG, EMG, ERG, respiratory functions, ABPM, photoplethysmography, oxymetry, phonocardiography, etc. Certain example embodiments may further provide for non-invasive, cost-effective, user friendly, continuous monitoring of complex multisystemic vital functions such as, polysomnography, electro mechanical coupling in the heart, neuro/cardiac interactions etc.

Certain example embodiments include the novel capability to display the full range spectral frequency, from a single beat, to scores of thousands of heart beats according to the length of the recording and limited only by the size, resolution, and fidelity of the screen chosen for the display. Conventional ECG analysis does not include, and is not capable of, single beat frequency analysis as performed in certain example embodiments. The frequency analysis in conventional ECG analysis involves signal averaging. Conventional techniques typically averaging 150 or more normal beats to be able to do frequency pattern analysis of the single averaged beat. To do that, in conventional systems, all abnormal beats are manually deleted. In certain example embodiments, by performing single beat frequency analysis of all normal and abnormal beats, frequency changes in single beats are found before the pathologic beat presents. Such an outcome is not possible with signal averaging of conventional systems, because the beat-to-beat variability, when averaged, is blended and may disappear.

In some embodiments, visual pattern recognition of the combination of the different parameters simultaneously displayed, based on a library of predetermined patterns enables improved or optimum diagnostic interpretation of biological signals. Automated digital pattern recognition may be used in some embodiments.

According to some embodiments, the visually-compressed spectral frequency and morphology of single or tens of thousands of waves such as (but not limited to) heart beats, can be clearly displayed in one screen. Unlike conventional techniques, averaging of multiple heart beats after elision of abnormal beats is not required and is not desirable for ECG or other biological signal frequency analysis.

In some embodiments, because of exact time domain synchronization, the conventional morphology and a novel frequency of the ECG signal can be clearly visualized, measured, and interpreted.

Using special signal display controls tools (e.g., gamma control, selective power gain, frequency isolation tools), different characteristics of the waves displayed can be adjusted in embodiments to improve or optimize visualization of the signals including those of the several singular frequencies embedded within the traditionally visualized ECG. The single line conventional ECG harks back to the year 1902. The ECG morphology/timeline is the morphologic representation of all electrical forces being generated and conducted within the heart at any given moment. In contrast to the conventional ECG, the embodiments enable the dissection of the electrical forces that contribute to the generation of the single line ECG in a manner similar to what the computerized axial tomography does to reveal traditional X-ray components.

Using special frequency display settings, precise, potentially unmistakable, identification of important ECG landmarks for diagnostic interpretation of starting and ending points of each wave and segment can be performed in accordance with certain example embodiments.

In certain example embodiments, waves and segments of waves in the time domain can be precisely measured having spectral display, in the single beat mode, as a controlling parameter to detect presence, change, or absence of intra-cardiac electrical currents. Interval and wave measurements can be done beyond microsecond precision. The ultrahigh fidelity of the signal recorded which is processed to preserve all the nuances of the original signal enables precise measurements.

In some embodiments, customized settings can be used for enhanced exact isolation of permanent and de novo (e.g., newly occurring) transient frequencies hidden within and composing the traditional ECG wave. The exact time of onset and offset of the de novo frequencies can be correlated with the morphologic, waves and segment changes in the traditional ECG.

In some embodiments, the power variations in each frequency are visualized as a respective color band (e.g., over a black=0 energy background) that changes in luminance, tonality, and/or saturation as the power within the band changes. Power can be readily measured using a variety of units.

The frequency display pattern can be also seen in a tridimensional display that facilitates rapid finding of certain pathologic features such as complex arrhythmia beats.

In some embodiments, the frequency range can be measured below 1 Hz, and the amplitude of each frequency band can be readily visualized. It is noted that markers for ventricular repolarization reserve (deadly arrhythmia risk) are usually found below the 10 Hz range.

Identification of electrocardiographic signs of pathology, especially those of abnormal atrial and ventricular repolarization (the predisposing factor for potentially deadly arrhythmia) are made possible by embodiments, for example, based upon recording and protecting the signals encoded in the low frequency range. Preferential attention to the low frequency is not done in currently used digital ECG which is QRS (high frequency) centered as it has been for over a century.

Conventional processing focuses on the QRS complex (high frequency) of the ECG. It fails to protect the ECG low frequency spectral range where important signs of cardiovascular risk are encoded.

In some embodiments, pitch changes in the fundamental frequency denote heart rate and likely other variations, and are a useful guide for quick location of pathologic waves even if tens of thousands of heart beat complexes are simultaneously displayed via one computer screen.

Sporadic or permanent changes in the pattern of the fundamental frequency, and its harmonics, within and between different ECG leads displayed according to some embodiments, lead to regions of wave changes of diagnostic significance.

In certain example embodiments, the fundamental as well as the frequency bands of interest, and their corresponding harmonics (if so desired) can be isolated, and color-coded for easy identification of their location within the traditional ECG morphology. The color-coded isolated frequencies and harmonics can also be superimposed for comparison of their relative importance at any given time prior, during, or after signal changes of potential diagnostic interest to detect myocardial functional and structural changes.

Independent power gain control of each isolated frequency can be used, e.g., for easy identification of their role and location within the traditional ECG signal.

Certain example embodiments enable the easy visualization ECG in the frequency display panel of undesirable noise that frequently obscures diagnostic features in the conventional displays, and the potential rapid elimination thereof.

Moreover, certain example embodiments enable annotation of the views with precise markers and text included in the displayed panels, e.g., in order to identify points of diagnostic interest. This annotation technique may be user-directed and/or automatic, in whole or in part. Screenshots of the total or partial display are used in some embodiments to document diagnosis and to be included in the summary report.

The example signal analysis techniques described herein may be performed in some embodiments by rapidly shifting between different levels of visual compression and/or recorded leads.

Some embodiments may be used, while the signal streams in real time in a screen, to visualize and analyze biological signals during a proprietary, standardized, short-term, physical emotional and physiological stress test protocol performed at a primary care facility. The results can then be used to guide as to continue the recording in ambulatory bases for days or weeks.

Real time signal monitoring can also be effectively used in standardized stress testing of different modalities as well as monitoring in intensive care and emergency settings, telemedicine, etc.

Example embodiments may be particularly beneficial in healthcare delivery, all physicians who need to obtain an ECG—the most frequently done medical test carried out in a doctor's practice- to get appropriate, fast, cost effective diagnosis. Example embodiments may also be particularly beneficial in the new drug development. For example, embodiments may provide interim, objective, sensitive and specific endpoints to validate the efficacy and safety of new drugs, thereby decreasing both the time and the cost associated with bringing a new drug to market.

Although certain example embodiments of the disclosure have been described, certain variations and modifications will be apparent to those skilled in the art, including embodiments that do not provide all the features and benefits described herein. It will be understood by those skilled in the art that the present disclosure extends beyond the specifically disclosed embodiments to other alternative or additional embodiments and/or uses as well as obvious modifications and equivalents thereof. In addition, while a number of variations have been shown and described in varying detail, other modifications, which are within the scope of the present disclosure, will be readily apparent to those of skill in the art based upon this disclosure. It is also contemplated that various combinations or sub-combinations of the specific features and aspects of the embodiments may be made and still fall within the scope of the present disclosure. Accordingly, it should be understood that various features and aspects of the disclosed embodiments can be combined with or substituted for one another in order to form varying modes of the present disclosure. Thus, it is intended that the scope of the present disclosure herein made should not, in any way, be limited by or to the particular disclosed embodiments described above.

1. A computer-implemented medical diagnosis method of analyzing a non-stationary non-linear biological signal recorded via one or more probes attached to a patient and stored in a memory, comprising:
accessing the stored recording of the biological signal in the memory;
determining a region of the recording to be displayed;
obtaining a signal of a morphologic characteristic and a signal of a spectral frequency characteristic included in the recording;
displaying for the determined region of the recording, spatially-separated and synchronized in time with each other, a first visually-compressed view including the signal of the morphologic characteristic and a second visually-compressed view including the signal of the spectral frequency characteristic; and
upon receiving an input from a user, changing a degree of visual compression of the displayed first visually-compressed view and displayed second visually-compressed view.

2. The computer-implemented method according to claim 1, wherein the determined region of the recording extends for plural hours, days, or weeks.

3. The computer-implemented method according to claim 1, wherein the displaying comprises displaying the first visually-compressed view and the second visually-compressed view on a same display screen.

4. The computer-implemented method according to claim 1, further comprising:
upon receiving the input from the user, determining a second region of the recording based upon the received input; and
performing said changing the degree of visual compression in accordance with the second region of recording.

5. The computer-implemented method according to claim 4, wherein the second region of the recording is shorter than the first region, and the first visually-compressed view and the second visually-compressed view are both less visually compressed after the changing.

6. The computer-implemented method according to claim 1, wherein the first visually-compressed view does not include averaged signal values of the morphologic characteristic and the second visually-compressed view does not include averaged signal values of the spectral frequency characteristic.

7. The computer-implemented method according to claim 6, wherein the biological signal is an electrocardiogram (ECG) signal.

8. The computer-implemented method according to claim 1, wherein the biological signal comprises at least one of electrocardiogram (ECG) signal, electroencephalogram (EEG) signal, electromyogram (EMG) signal, electro-retinography (ERG) signal, signal corresponding to respiratory functions, signal corresponding to ambulatory blood pressure (ABPM), signal generated by photoplethysmography, signal generated by oxymetry, or signal generated by phonocardiography.

9. The computer-implemented method according to claim 1, wherein the displaying includes displaying power variations in each frequency using color bands that change in luminance, tonality, and saturation as the power level within the band changes.

10. The computer-implemented method according to claim 1, wherein the displaying includes displaying selected frequency bands in configurable color and/or contrast in order to identify their respective contributions to the morphologic structure of the biological signal.

11. The computer-implemented method according to claim 1, further comprising annotating the displayed first visually-compressed view and the displayed second visually-compressed view with markers and text to mark waves or wave components of diagnostic interest.

12. The computer-implemented method according to claim 1, further comprising providing for rapidly shifting between different levels of visual compression and/or biological signal recording leads for said displaying of the first visually-compressed view and the second visually-compressed view.

13. The computer-implemented method according to claim 1, further comprising providing screenshots of the display screen captured when displaying the first visually-compressed view and the second visually-compressed view at the determined region of recording and at the second region of recording.

14. The computer-implemented method according to claim 1, wherein the displaying comprises displaying at least one of the first visually-compressed view or the second visually-compressed view in three-dimensions.

15. The computer-implemented method according to claim 1, further comprising displaying automatically or manually identified normal and abnormal, transient and permanent frequencies hidden within and composing the acquired biological signal.

16. The computer-implemented method according to claim 15, further comprising displaying segregated frequencies of diagnostic interest.

17. The computer-implemented method according to claim 16, wherein the segregated frequencies are color coded for identification of their respective contributions to the morphology of the biological signal.

18. The computer-implemented method according to claim 1, further comprising superimposing on at least one of the displayed first visually-compressed view or the displayed second visually-compressed view aspects from a library of predetermined biological patterns.

19. A medical diagnosis system for analyzing a non-stationary non-linear biological signal recorded via one or more probes attached to a patient, comprising:
a memory configured to store the recorded biological signal; and
at least one processor configured to perform operations comprising:
accessing the stored recording of the biological signal in the memory;
determining a region of the accessed recording to be displayed;
obtaining a signal of a morphologic characteristic and a signal of a spectral frequency characteristic included in the accessed recording;
displaying for the determined region of the accessed recording, spatially-separated and synchronized in time with each other, a first visually-compressed view including the signal of the morphologic characteristic and a second visually-compressed view including the signal of the spectral frequency characteristic; and
upon receiving an input from a user, changing a degree of visual compression of the displayed first visually-compressed view and displayed second visually-compressed view.

20. The system of claim 19, further comprising a display screen, wherein the first visually-compressed view and the second visually-compressed view are displayed via the display screen.

21. The system of claim 19, further comprising a recorder electrically connected to the one or more probes and configured to acquire the biological signal.

22. A non-transitory computer-readable storage medium storing a computer program for analyzing for medical diagnosis a non-stationary non-linear biological signal recorded via one or more probes attached to a patient, the computer program, when executed by a processor, causes the processor to perform operations comprising:
accessing the stored recording of the biological signal in the memory;
determining a region of the recording to be displayed;
obtaining a signal of a morphologic characteristic and a signal of a spectral frequency characteristic included in the recording;
displaying for the determined region of the recording, spatially-separated and synchronized in time with each other, a first visually-compressed view including the signal of the morphologic characteristic and a second visually-compressed view including the signal of the spectral frequency characteristic; and
upon receiving an input from a user, changing a degree of visual compression of the displayed first visually-compressed view and displayed second visually-compressed view.
